(19) United States
(12) Patent Application Publication
(10) Pub. No.: US 2003/0044785 A1
(43) Pub. Date: Mar. 6, 2003

(54) Method for identifying and genotyping microorganisms

(75) Inventor: Mark S. Berninger, North Potomac, MD (US)

Correspondence Address: ROTHWELL, FIGG, ERNST & MANBECK, P.C., 1425 K STREET, N.W., SUITE 800, WASHINGTON, DC, 20005, US

(73) Assignee: American Type Culture Collection, Manassas, VA (US)

(21) Appl. No.: 09/845,320

(22) Filed: May 1, 2001

(51) Int. Cl. C12Q 1/68; C12P 19/34

(52) U.S. Cl. 435/6; 435/91.2

(57) ABSTRACT

A method for rapidly determining the species and/or strain of an organism by genotyping is presented. The method extends the use of indexing linkers to a method of genotyping. The genome of an unknown organism is digested by a restriction endonuclease which cleaves at a site different from the recognition site of the enzyme thereby producing DNA fragments and producing staggered ends. A subset of these fragments is ligated to linkers with staggered ends, with only those DNA fragments with staggered ends which are complementary to the staggered ends of the linkers being ligated to the linkers. DNA fragments with linkers at each end are then amplified. The number and sizes of amplified DNA fragments are then compared to a database containing the expected number and sizes of fragments from known organisms. A match between the assay data and the database is determinative of the species or strain of the unknown organism. In a preferred mode, the assay utilizes a single enzyme, single linker and single primer thereby resulting in a simplified assay.

### BACKGROUND OF THE INVENTION

Many different methods of identifying microorganisms have been used over the years. The most recent methods are based upon DNA based genotyping. These methods identify or “type” organisms based upon their genomic characteristics. The most complete genotyping method would be to sequence completely the genome of the organism to be identified and to compare this sequence to the complete sequences of other organisms. Such a method is currently far too time consuming and costly.

A collection of methods generally known as “DNA fingerprinting” has been widely used to identify organisms. These methods can be utilized to distinguish between organisms of different species and also to distinguish between organisms within a single species. In all these methods multiple genetic loci are interrogated to ascertain which of two or more alleles are present. In general, the more alleles there are that are variable in the population from which the organisms that are interrogated are taken, the simpler it is to distinguish between organisms that are closely related genetically. In some instances it is unnecessary to distinguish between closely related organisms, whereas in other instances a higher level of discrimination at the genetic level between organisms is needed. The more closely related two organisms are genetically, the more genetic loci must be interrogated. Accordingly, methods that permit one easily to modulate the number of genetic loci being interrogated are of value.

One method, Restriction Fragment Length Polymorphism (RFLP), comprises digesting the genomic DNA of the organism with restriction endonucleases to form DNA fragments, separating the DNA fragments electrophoretically on a gel, transferring the DNA fragments to a membrane and hybridizing the separated fragments with a specific probe to detect a specific subset of fragments that result from the restriction endonuclease digestion. In this method, differences in sizes of the hybridized fragments indicate allelic differences between the genomes of different organisms. If necessary, different and additional restriction endonucleases and/or different probes can be employed to interrogate more loci. The pattern of fragments hybridized can be analyzed to determine the species or strain of an organism being tested by comparing the pattern against known patterns within a database. In this method, to increase the number of loci being interrogated, additional reagents, restriction endonucleases and/or hybridization probes that require differing reaction conditions must be used.

Several different methods of genotyping in addition to restriction endonuclease RFLP have also been developed. These methods, like RFLP, rely on analyzing a subset of DNA fragments obtained from the genomic DNA of the organism under study.

One such method, amplified fragment length polymorphism (AFLP) analysis is commonly used to genotype organisms, most commonly plants (Vos et al., 1995; EP0534858; U.S. Pat. No. 5,874,215). This method has also been applied to the analysis of bacteria (Ripabelli et al. (2000) and Willems et al. (2000)). In this method genomic DNA of an organism is digested with one or more Type II restriction endonucleases to produce DNA fragments. Double-stranded adaptors are then ligated to the DNA fragments. After ligation of the adaptors, a polymerase chain reaction (PCR) is performed using primers which are complementary at their 3′ ends to only a fraction of the fragments to which adaptors were ligated. PCR methods are widely known by those of skill in the art. See, e.g., Innis et al. (1990). Since efficient initiation of PCR is dependent on base pairing at the 3′ end of the primer, PCR only is carried out most efficiently at those sites where the 3′ sequence of the primer being used is complementary to the sequence of the particular fragment. Since sequences adjoining Type II restriction endonuclease cleavage sites vary essentially independently from the cleavage site, only a fraction of restriction endonuclease fragments efficiently amplify. This subset of fragments is then separated electrophoretically and visualized in a number of ways. Alternatively the DNA could be labeled such as with a fluorescent or radioactive label which was incorporated in the PCR primers, thereby aiding in subsequent visualization. By varying the sequence of the primers, especially the sequence of the 3′ end of the primer, and their number, more or fewer fragments can be amplified. Since each fragment represents interrogation of a particular potentially variable genomic locus, the number of loci examined can be varied by varying the numbers of fragments amplified. To distinguish among closely related organisms, many loci must be interrogated, whereas to distinguish among more distantly related organisms fewer need be examined. In this method, specificity of primer elongation is critical to obtaining analyzable and reproducible data and specificity of primer elongation requires precise control over the temperature and ionic conditions of the elongation reaction. Controlling these parameters can be challenging especially when many samples are tested at a time or at different times. It is especially challenging to have different laboratories reproduce these conditions exactly.

Another method, called Random Amplified Polymorphic DNA Analysis (RAPD), involves the use of short (approximately 10 nucleotide long) primers randomly to amplify fragments of genomes, typically genomes of several megabases. The reproducibility of this method rests critically on having exactly reproducible conditions for amplification. In practice this is difficult to accomplish, particularly between different laboratories (McDowell (1999) and Saunders and Hopkins (1999)).

Other methods for fingerprinting genomes have been developed that are based on amplifying regions of the genome that are known to be highly variable among individuals in a population. These methods have the advantage of being highly informative, especially in a forensic context, but have the disadvantage of being predicated on foreknowledge of considerable molecular genetic information about the organism being genotyped. Such methods cannot easily and rapidly be applied to new organisms about which little or no molecular genetic information is available.

A method has been developed that permits one to select biochemically a subset of regions of sequence from a genome and to make that subset of regions amplifiable by PCR. This method utilizes indexing linkers (U.S. Pat. Nos. 5,508,169; 5,858,656). In this method a genome is digested with a Type IIS restriction endonuclease or with a restriction endonuclease that cleaves within an interrupted recognition sequence. Both types of enzymes recognize a specific sequence of nucleotides but cut at a site other than the recognition sequence. Several of these endonucleases leave fragments that have short cohesive termini, often termed “sticky ends”. The sequence of such a cohesive terminus may be, and usually is, different than the nucleotide sequence recognized by the restriction endonuclease. For any particular cleavage site in a genome the sequence of the cohesive end generated by cleavage at that particular site has a particular sequence, however the various cleavage sites in the genome will, when digested, lead to a variety of cohesive end sequences. Therefore taken as a group the cohesive ends generated by digestion of a genome will be varied, but for any particular cleavage site the cohesive end will not be variable. For example, Fok I recognizes the sequence 5′-GGATG-3′ but cuts nine bases 3′ away on one strand and thirteen bases 5′ away on the complementary strand thereby leaving a four base cohesive terminus. The sequence of the resulting four base overhang will vary from fragment to fragment, but for each particular fragment end the cohesive terminus will have a particular cohesive end. Bgl I is an example of a restriction endonuclease that cuts within an interrupted palindromic sequence. Bgl I recognizes the sequence 5′-GCCNNNNNGGC-3′ (SEQ ID NO: 1) with the enzyme cleaving between the two most 3′ Ns on each strand thereby leaving a three base overhang which has a variable base composition. In the indexing method, linkers are ligated to the restriction endonuclease digested nucleic acid. Again, it is desired that only a subset of the fragments be ligated to the indexing linkers. To accomplish this, only specific linkers are utilized. For example, if Bgl I is used to digest the DNA, a three base overhang can be any one of 64 possible base sequences. By using indexing linkers with a 3′ overhang with only one of the 64 possible base sequences, on average only 1/64th of the ends will be ligated to the indexing linker. Assuming that the two ends of a fragment vary independently, a reasonable assumption, then on average only approximately 1/4096 of fragments will have an indexing linker ligated to each end. Variations upon the number and types of restriction endonucleases utilized and the type of linker used as well as the number of bases in the overhang region will result in varying subsets of the DNA fragments being ligated at each end to an indexing linker. The DNA sample is then amplified using PCR wherein the primers are complementary to a portion of the indexing linkers used. Only those fragments with the proper linkers at each end will be exponentially amplified and observed. The indexing linker method has been applied to the selective isolation of nucleic acid fragments containing only one of all the possible cohesive ends, identification of the exact sequence of bases present in the cohesive ends of each subset of fragments present alone or in a mixture, selective amplification by the polymerase chain reaction of fragments containing only one or two of all such possible cohesive ends without knowledge of the base sequence of their double stranded portions, selective labeling of one strand, or one end, of one or more subsets of fragments with indexing linkers containing detectable reporter groups, selective modification of one end, or one strand, of one or more subsets of fragments containing such cohesive ends to enable or disable the action of various enzymes which can act on nucleic acids such as polymerases, polynucleotide kinases, polynucleotide ligases, exonucleases, or restriction endonucleases, and rapid determination of restriction endonuclease maps of fragments cleaved with restriction endonucleases which reveal such cohesive ends (U.S. Pat. Nos. 5,858,656; 5,508,169).

A variety of methods of analyzing the genomes of organisms have been developed. These include random amplified polymorphic DNA (RAPD) (Williams et al., 1990), arbitrarily primed PCR (AP-PCR) (Welsh and McClelland, 1990; Welsh and McClelland, 1991) and DNA amplification fingerprinting (DAF) (Caetano-Anolles et al., 1991). Other DNA fingerprinting techniques are taught by Jeffreys et al., 1985; Nakamura et al., 1987; Jeffreys et al., 1991; Tautz, 1989; Weber and May, 1989; Edwards et al., 1991; Beyermann et al., 1992; and Brenner and Livak, 1989.

Each method of genotyping or fingerprinting has its advantages and disadvantages with some being more suitable for small genomes and others being more suitable for large genomes. Each of these methods relies on the specificity of primer binding and elongation to generate a specific and limited subset of amplifying fragments. This specificity of amplification is highly dependent on controllable and reproducible thermal conditions during PCR. Such reproducibility is difficult to achieve in practice and is especially difficult to achieve between laboratories. Despite the wide number of methods in use, there is still a desire for a simpler and more robust and reproducible method of genotyping microorganisms.

A typical bacterium may have a genome of about two to several million basepairs. Humans have a haploid genome of approximately three billion basepairs. When these genomes are digested with a restriction endonuclease the number of fragments which are formed is very large. Even for a bacterium with a genome of only two million basepairs, an enzyme which has a four base palindromic recognition sequence will on average produce approximately 8,000 fragments of DNA. If a restriction endonuclease with a five base non-palindromic recognition sequence is used then on average a two million basepair genome will be digested into approximately 4000 fragments. If a restriction endonuclease recognizes a five base palindromic sequence, then approximately 2000 fragments will result. If a restriction endonuclease with a six base non-palindromic recognition sequence is used then approximately 1000 fragments will be produced. Even 1000 fragments are too numerous to easily analyze and it is likely many bands will overlap making analysis extremely difficult. Therefore most methods of genotyping that use restriction endonuclease digestion include a means to analyze only a subset of the fragments which are produced. In some methods, one or more specific probes were utilized so that only one or a few fragments would be observed and the size of those fragments would be determined.

The publications and other materials used herein to illuminate the background of the invention, an in particular, cases to provide additional details respecting the practice, are incorporated herein by reference, and for convenience, are referenced by author and date in the text and respectively grouped in the appended List of References.

### SUMMARY OF THE INVENTION

The invention is directed to a method to genotype rapidly organisms, most especially microorganisms, thereby determining the species and even strain of the organism. The method comprises digesting the genome of the microorganism with a restriction endonuclease that either cleaves at a distance from the recognition site or cleaves within an interrupted recognition sequence. The ends of the DNA fragments produced by such enzymes will have several different DNA sequences and not a single sequence such as produced by a Type II restriction endonuclease which cleaves within its recognition site. Linkers are ligated to the ends of the DNA fragments produced by the restriction endonuclease digestion. The linkers are limited such that they have specific ends so that only the subset of all of the DNA fragments which has extensions complementary to the linkers can be ligated to the linkers. After the linking step the fragments are amplified such as by a polymerase chain reaction utilizing primers complementary to the linkers. The amplified fragments are identified according to size and this restriction digest information is compared to previously prepared databases of restriction fragments of genomes of organisms using the same enzymes. Data can be obtained using a single restriction endonuclease and a single linker, thereby simplifying the assay as compared to other methods of genotyping, although it may be necessary to utilize more than one enzyme and/or more than one linker to generate more amplifiable fragments and thereby to be able to distinguish between closely related species or strains of microorganisms.

### DETAILED DESCRIPTION OF THE INVENTION

The invention is directed to a method of quickly and easily identifying the species and strain of a microorganism. It is based upon the Indexing Linkers method as described by Deugau and Unrau (U.S. Pat. Nos. 5,858,656; 5,508,169; Unrau and Deugau, 1994). The indexing linkers method relies upon a method of cleaving genomic DNA with a restriction endonuclease such as a Type IIS enzyme which cleaves at a distance from the recognition sequence. The ends of the DNA fragments formed with such an enzyme are variable. For example, Fok I cleaves one strand nine bases from the recognition sequence in one strand and thirteen bases from the recognition sequence in the complementary strand thereby leaving a four base overhang. The sequence of four bases can be anything, there being 4⁴ or 256 possible sequences of this overhang. Linkers are ligated to the ends of the DNA fragments formed from the restriction digestion. If only Fok I were used in the digestion and only a single linker were used with one of the 256 possible sequences, then only one out of every 256 ends would ligate and only one out of every 256² (65,536) fragments would have a linker at each end. To increase the number of fragments with linkers at each end, a second linker and/or second enzyme can be included in the assay. The fragments with linkers at each end are amplified using primers complementary to the linkers. The method results in the visualization of only a subset of the total number of fragments formed by the restriction digestion. Depending upon the size of the genome, the fraction of fragments which is visualized can be altered by utilizing enzymes which leave either a 3, 4 or 5 base overhang. The selection of the subset of fragments observed is dependent upon the sequence of the overhanging ends of the indexing linkers which are used.

In the AFLP method, the selection of the subset of fragments which is observed is obtained by a different method. Typically in the AFLP method a genome will be digested by one or more Type II restriction endonucleases which cut within the recognition sequence with each enzyme leaving only one discrete overhang. The bases neighboring the recognition site of the restriction endonuclease will be variable. After digesting the genomic DNA, linkers are attached with the linker overhangs being 100% complementary to the DNA fragment overhangs thereby resulting in all fragments receiving linkers. The fragments are then amplified using primers which are complementary to the linkers but which extend by one or more bases into the region of the fragment beyond the restriction endonuclease recognition sequence. Only those fragments which are complementary to the primers, particularly at the 3′ end of the primer, will amplify. If a primer extends one base beyond the recognition site, on average only one in four fragment ends will be complementary at the 3′ end of the primer. Considering both ends, only one in sixteen fragments will have both ends complementary to 3′ ends of the primer or primers being utilized. Therefore only 1/16 of the fragments will efficiently amplify. If the primers are designed to extend two bases beyond the recognition sequence an even smaller fraction of the fragments would be sufficiently complementary at the 3′ ends of the primers to amplify. The added bases at the ends of the amplification primers are called selective nucleotides. The AFLP method therefore uses selective nucleotides which must be complementary to an internal region of the DNA fragments as the means for selecting a subset of fragments to be visualized. Furthermore, AFLP relies on the specificity of primer elongation in PCR to select those fragments that amplify and, through this selection, limits the number of fragments being analyzed.

The present invention employs elements of the Indexing Linker method but extends and improves that method to make it useful for quickly determining the identity of an organism, especially a microorganism. Furthermore, whereas the Indexing Linker method is to be performed utilizing two or more restriction endonucleases and a protruding end of 3, 4 or 5 bases, the present invention preferably utilizes a single restriction endonuclease and linkers which are complementary to cohesive ends having only two bases.

The invention is a fingerprinting or genotyping technique which preferably utilizes a single restriction endonuclease, single linker and single amplification primer thereby simplifying the assay. Further this invention enables one to use the same single restriction endonuclease, single linker and single amplification primer to analyze a multitude of different organisms. Further, this invention requires no prior molecular genetic knowledge of the organism(s) being analyzed; in particular, it is unnecessary to have sequenced any of the genome of the type of organism being analyzed. In brief, the organism is identified by cleaving the genomic DNA with a restriction endonuclease, attaching linkers to the ends of the DNA fragments which are formed, amplifying a subset of the DNA fragments, and comparing the pattern of sizes of the amplified subset with databases of restriction fragments observed with organisms treated with the same enzymes, linkers and primers. It is desirable to visualize only a subset of the fragments because in general too many fragments are produced by restriction endonuclease cleavage of a complete genome, the large number of fragments inhibiting the ability to analyze the data. For example, using a restriction endonuclease which recognizes a four base palindromic sequence to digest a genome of 2×10⁶ basepairs would on average yield approximately 8,000 fragments. If all of these fragments were electrophoresed on a gel and visualized it is likely that a smear would be seen rather than discrete bands. Consequently it is necessary as a practical matter to limit the visualization to only a subset of fragments. Use of a restriction endonuclease with a 5-base or 6-base non-palindromic recognition site would limit the number of bands to approximately 4000 or 1000 fragments, respectively. Of course, microorganisms with larger genomes would have correspondingly more fragments, e.g., an organism with a 4×10⁶ basepair genome would yield approximately 16,000, 8000 or 2000 fragments depending on the restriction endonuclease used. These numbers are all too high to easily analyze or even to obtain useful data. Means to view a subset of the fragments must be utilized.

In the invention, genomic DNA from the organism to be identified is digested with a Type IIS restriction endonuclease or with a restriction endonuclease which cleaves within an interrupted palindromic sequence with the enzymes cutting in a staggered fashion thereby leaving one strand with an extension. With either type of enzyme the DNA fragments produced will have a variety of base sequences in the protruding extensions. Preferably a restriction endonuclease with a five base recognition sequence is utilized to analyze microorganisms because, for microorganisms with a genome on the order of one to several million basepairs, it will result in the formation of a number of DNA fragments which will yield enough information to be useful and not so many fragments that the identification of individual fragments will be difficult. Use of other enzymes is possible but not preferred, e.g., using an enzyme which recognizes a six base or longer sequence may result in the formation of too few DNA fragments to be useful whereas use of an enzyme which recognizes a four base site may produce a number of DNA fragments which is too large to be easily evaluated even after linker indexing is used to reduce the number of amplifiable fragments. Additionally, use of an enzyme having five-base recognition sequences will result in fragments of a size range that is easy to analyze.

In addition to using a restriction endonuclease which preferably recognizes a five base sequence, it also is preferable that the restriction endonuclease produce a staggered cut leaving a two base overhang which can act as a cohesive end. With a two base overhang there are sixteen possible dinucleotide cohesive ends. Each fragment may have both ends with identical or with different dinucleotide cohesive ends. Assuming a random distribution of cohesive terminal sequences, then if a certain fragment has, for example, the sequence CC at its “left” end (approximately one in 16 fragments) it also has a one in 16 chance of having a CC at its other end, leading to the calculation that on average one in 256 fragments will have CC at both termini. Since a restriction endonuclease that cleaves at a specific five base long non-palindromic site cleaves complex DNA roughly once every 500 bases, the total number of fragments generated by complete digestion of a two megabase bacterial genome will be approximately 4000. If one in 256 has, for example, a CC at both ends, then only about 16 such fragments having a CC at both ends will exist in the digested genome. These dinucleotide cohesive ends can be ligated with a linker which has a corresponding GG two base cohesive end. The linker can be any arbitrary sequence of nucleotides but should be long enough to be complementary to a useful primer for performing an amplification such as PCR. If the subset of DNA fragments to be examined have identical dinucleotide cohesive ends at both ends (i.e., both ends are CC, both ends are AC, etc.) then only a single type of linker needs to be utilized and it will link at each end of the fragments to be visualized. It should be noted that since it is necessary to add a linker using a dinucleotide complement, it is preferred that dinucleotide cohesive ends which can circularize are not utilized. For example, preferably one would not select to visualize the subset of DNA fragments which have a GC dinucleotide cohesive end at both ends because the fragments could circularize or add linkers and the addition of linkers to such molecules could be less efficient. Further, the linkers used in this example would be mutually complementary and would ligate to form linker dimers that could be very efficiently amplified in a PCR, thereby competing with amplification of restriction endonuclease generated fragments.

It should be noted that identical reagents (enzymes, linkers, primers, etc.) can be utilized regardless of the organism being studied allowing for simplified and less costly assays since only a single set of reagents is required regardless of the organisms to be studied.

The use of two base cohesive termini is preferred because the ligation of a two base overlap is especially specific.

Fragments to be visualized can then be amplified by any of several known amplification schemes, e.g., a polymerase chain reaction can be used. In the case of use of a single linker, both ends of the desired fragments are identical since they both have an identical linker attached. A single primer can be used to amplify the desired subset via PCR since the primer is complementary to both ends. Only those fragments which contain linkers at each end will be exponentially amplified and visualized. Fragments containing no linkers will not be amplified at all and fragments containing a linker at a single end will be only linearly, not exponentially, amplified and will not be detected. The result of this hypothetical example will be amplification of approximately 16 fragments of DNA which will be visualized and these fragments should center around approximately 500 basepairs which is a useful size range for performing amplification such as by PCR. The lengths of the individual bands will depend upon the arrangement of particular restriction endonuclease cleavage sites and the sequence of the cohesive termini generated following cleavage. The sizes of each of the approximately 16 fragments can be determined, e.g., by electrophoresis on a gel, although other methods known to those of skill in the art for size separating nucleic acids may be used. The fragments can be labeled if desired. Many methods of labeling the fragments are known to those of skill in the art. One preferred method is to utilize PCR primers which include a label such as a fluorescent marker or a radioactive marker.

Once the number of fragments and the size of each fragment are determined, these data may be compared to a database containing information derived by the application of this method to the genomes of other organisms which have been digested with the same restriction endonuclease and which have had DNA fragments ending with the same dinucleotide cohesive ends selected. In this way one may determine whether the organism being analyzed is the same, or is closely related to, a previously analyzed organism. In a preferred method the database is stored in a computer and the data to be analyzed is analyzed with the use of a computer. Generally when using this method, the more closely related two organisms are the fewer differences there will be which are observable between the amplified fragments which are generated. An identical match of the number of DNA fragments together with the size of each fragment (adjusted as necessary to take into account the size of the linkers which are attached) may be used to identify effectively the species and subspecies of the organism. As necessary, more fragments can be analyzed by repeating the analysis using a different restriction endonuclease and/or using different cohesive termini to which linkers are ligated, distinguishing among more closely related organisms. This capability may be especially valuable in epidemiological surveillance of infectious outbreaks, particularly hospital-acquired infections, to detect genetic regions associated with antibiotic resistance and/or pathogenicity.

If the number of fragments visualized is larger than expected, this is an indication that the sample being assayed may contain more than a single type of organism, e.g., the sample may be contaminated, although this is not necessarily true since by chance a restriction site may appear an unexpectedly high number of times within a single genome.

The method is not limited to visualizing only those DNA fragments which have identical dinucleotide cohesive ends at both ends. Fragments with different dinucleotide ends can also be utilized although such an approach is slightly more complex and expensive. A variety of approaches can be used. For example, if one wants to visualize the subset of DNA fragments with one CC cohesive end and one AC cohesive end, as well as fragments having CC at each end or AC at each end, two different linkers would be utilized, with one having a cohesive end complementary to CC and the other having a cohesive end complementary to AC. The two linkers could also have the same arbitrary sequences such that a primer which primes on one linker will also prime on the second linker. This method does require the use of two different linkers but only one primer. It also results in the visualization of more bands in a single assay as compared to an assay which visualizes only DNA fragments with identical ends and a single primer. This single assay using two linkers but only one primer which amplifies multiple subsets at a single time may be useful if more bands are required to distinguish closely related species or strains. It should further be appreciated that all linkers, regardless of the cohesive ends, can incorporate the identical arbitrary sequence which will be complementary to the primer to be used. If this is done, only a single universal primer need be used regardless of which subset of DNA fragments is to be examined. The specificity of the analysis is not primarily based on specific elongation of primers, rather it is due primarily to the specificity of restriction endonuclease digestion and to the specificity of ligation of the staggered, cohesive ends of the linkers which are used to attach the priming sequences to the fragments.

Two Type IIS restriction endonucleases which are preferred for performing the assay are BstF5 I and Fau I. Each of these enzymes recognizes a five basepair sequence and cleaves the DNA leaving a variable two base overhang or cohesive end. BstF5 I recognizes and cleaves DNA as:

5′ . . . GGATGNN\hat{ } . . . 3′
3′ . . . CCTAC\hat{ }NN . . . 5′

and Fau I recognizes and cleaves DNA as:

| | |
|---|---|
| 5′ . . . CCCGCNNNN\hat{ } . . . 3′ | |
| 3′ . . . GGGCGNNNNNN\hat{ } . . . 5′ | (SEQ ID NO:2) |

where indicates the cleavage site within each strand.

Although the use of a restriction endonuclease which has a 5 basepair recognition site will usually be preferred, other restriction endonucleases may be used, especially in the case of an unusually small or unusually large genome or if the genome has an unusual base composition which results in too many or too few bands when using a 5 basepair recognition enzyme.

Similarly, although it is preferred to use restriction endonucleases which cleave to form a two base overhang, it is possible to use longer overhangs and therefore broaden the number of possible enzymes which can be used. For example, use of a restriction endonuclease which has a 4 basepair recognition site will lead to a total number of fragments roughly four times as great as when a 5 basepair recognition sequence enzyme is used. But if the 4 base recognition enzyme leaves a 3 base overhang, the use of linkers with three base overhangs results in only ¼ the number of ligations as with a two base overhang thereby compensating for the overall larger number of fragments produced. However, as stated above it is preferred to use dinucleotide overhangs because they result in less background thereby yielding cleaner results.

To fully employ this method, it is necessary to create a database or databases against which subsequently obtained data may be compared. Such databases are easily obtainable by performing the assay as described on known organisms using a specified restriction endonuclease or restriction endonucleases and linkers with specified cohesive ends. Alternatively, as whole genomes of organisms are sequenced, the expected results can be determined directly from the known sequence. For such fully sequenced organisms it will be unnecessary to perform assays to determine the restriction fragment digestion pattern which will be seen for purposes of preparing a database.

### EXAMPLE 1

An unknown microorganism is cultured, nucleic acid is extracted and purified, and the nucleic acid is digested according to the supplier's recommended conditions by BstF5 I (New England Biolabs, Inc., Beverly, Mass.). The digested DNA is then annealed with and ligated to the oligomer 5′-TCACACAGGAAACAGCTATGACGG-3′ (SEQ ID NO:3) (Life Technologies, Rockville, Md.). Preferably on the order of 30 linker oligomers should be used per end of fragment after the restriction digestion. The number of ends will vary depending upon the amount of DNA present, the number of times the restriction site of choice appears within the genome, and upon the size of the genome. With unknown samples it will not be possible to determine an exact number of ends, but an estimate within one order of magnitude will be likely. One may prefer to perform more than a single ligation and PCR wherein the amount of linker varies, e.g., tubes with 1×, 3× and 10× amounts of the linker amount estimated to give 30 linkers per end. This particular oligomer anneals to fragments which have CC dimer ends as 3′ overhangs. One oligomer will anneal to a single end of fragments which have only a single CC overhang and oligomers will anneal to each end of fragments which have CC overhangs at each end of the fragment. After ligation, the nucleic acid is amplified with approximately 30 cycles of polymerase chain reaction using a thermostable polymerase such as Taq and amplification conditions appropriate to the primer sequence chosen. Selection of such amplification conditions is routine among practitioners of PCR. The primer to be used is identical to a portion of or all of the linker oligomer. In this case the primer is 5′-TCACACAGGAAACAGCTATGAC-3′ (SEQ ID NO:4). Preferably an elongation step is performed prior to the PCR to fill in the tails to make both strands completely double-stranded. With this single linker, only fragments with a CC overhang at each end of the fragment will be exponentially amplified and only these fragments will be seen on a stained gel. The amplified DNA is electrophoresed on a 2% agarose gel and stained with ethidium bromide. Marker lanes of DNA are simultaneously electrophoresed on the agarose gel and are used to determine the sizes of the fragments which are seen.

The DNA from the unknown microorganism can be electrophoresed on a gel with DNA from a known microorganism treated in the same manner as the DNA from the unknown microorganism, i.e., the DNAs are digested with the same restriction endonuclease, ligated with the same oligomers and amplified by PCR. If the sample from the unknown microorganism gives a pattern identical to that from the known microorganism, then it is known that the two microorganisms are either the same species or very closely related species, whereas if the pattern of DNA bands observed is different between the two, then it is determined that the two microorganisms are different species from each other.

### EXAMPLE 2

Because BstF5 I leaves a 3′ two-base overhang, it was possible to use a single-stranded oligomer linker, such as shown in Example 1. This single strand can hybridize via the two-bases of complementarity and the 3′ ends of the microbial DNA can be extended to give a fully double-stranded molecule which can be amplified. It is also possible, and is preferable, to use a double-stranded linker. The method of Example 1 is repeated, but rather than using a single-stranded oligomer, a partially double-stranded linker is used. This linker is:

| | |
|---|---|
| 5′-TCACACAGGAAACAGCTATGACGG-3′ | (SEQ ID NO:3) |
| 3′-AGTGTGTCCTTTGTCGATACTG-5′ | (SEQ ID NO:5) |

This double-stranded linker will anneal to the CC 3′ overhang and can efficiently be ligated to the microbial DNA. PCR is again performed using SEQ ID NO:4 as primer.

### EXAMPLE 3

The steps as in Example 1 are repeated except using an oligomer which is 5′-TCACACAGGAAACAGCTATGACCC-3′ (SEQ ID NO:6) (Life Technologies, Rockville, Md.) and the same primer as in Example 1 which is 5′-TCACACAGGAAACAG-3′ (SEQ ID NO:4). Using only this oligomer, only fragments which have a GG 3′ overhang at each end will be visualized.

### EXAMPLE 4

The steps as in Example 1 are repeated except that two different oligomers are included, these being SEQ ID NO:3 and SEQ ID NO:6. The primer is again SEQ ID NO:4. In this example, three different types of fragments will be visualized. The first is those fragments with a CC at each end (same as Example 1). The second group is those fragments with a GG at each end (same as Example 2). The third group includes fragments with a CC at one end and a GG at the second end. The use of two different oligomers together, as in this example, can be useful to increase the overall number of visualized bands, e.g., when one is dealing with a very small genome, when the genome by chance has relatively few restriction sites for the enzyme being used, when a restriction enzyme with a 6 base or longer recognition sequence is utilized, or when one needs an increased number of fragments to distinguish between very closely related species.

### EXAMPLE 5

An unknown microorganism is cultured, nucleic acid is extracted and purified, and the nucleic acid is digested according to the supplier's recommended conditions by Fau I (New England Biolabs, Inc., Beverly, Mass.). This digest leaves 2-base 5′ overhangs. In this Example, a double-stranded linker is used. The double-stranded linker is:

| | |
|---|---|
| 5′-GGTCACACAGGAAACAGCTATGAC-3′ | (SEQ ID NO:7) |
| 3′-AGTGTGTCCTTTGTCGATACTG-5′ | (SEQ ID NO:8) |

Preferably on the order of 30 linkers should be used per end of fragment after the restriction digestion. The number of ends will vary depending upon the amount of DNA present, the number of times the restriction site of choice appears within the genome, and upon the size of the genome. With unknown samples it will not be possible to determine an exact number of ends, but an estimate within one order of magnitude will be likely. One may prefer to perform more than a single ligation and PCR wherein the amount of linker varies, e.g., tubes with 1×, 3× and 10× amounts of the linker amount estimated to give 30 linkers per end. This linker anneals to fragments which have CC dimer ends as 5′ overhangs. One linker will anneal to a single end of fragments which have only a single CC overhang and linkers will anneal to each end of fragments which have CC overhangs at each end of the fragment. After ligation, the nucleic acid is amplified with approximately 30 cycles of polymerase chain reaction. Using conditions appropriate for amplification of 500-1500 basepair long fragments using Taq polymerase or other commonly used thermostable polymerases, such conditions being well known in the art. The primer to be used is identical to a portion of or all of SEQ ID NO:8. In this case the primer is 5′-GTCATAGCTGTTTCC-3′ (SEQ ID NO:9). With this single linker, only fragments with a CC overhang at each end of the fragment will be exponentially amplified and only these fragments will be seen on a stained gel. The amplified DNA is electrophoresed on a 2% agarose gel and stained with ethidium bromide. Marker lanes of DNA are simultaneously electrophoresed on the agarose gel and are used to determine the sizes of the fragments which are seen.

The DNA from the unknown microorganism can be electrophoresed on a gel with DNA from a known microorganism treated in the same manner as the DNA from the unknown microorganism, i.e., the DNAs are digested with the same restriction endonuclease, ligated with the same oligomers and amplified by PCR. If the sample from the unknown microorganism gives a pattern identical to that from the known microorganism, then it is known that the two microorganisms are either the same species or very closely related species, whereas if the pattern of DNA bands observed is different between the two, then it is determined that the two microorganisms are different from each other.

As was the case with Example 1, Example 5 can be varied by using a different linker or linkers and a different primer.

### EXAMPLE 6

Example 6 is similar to Example 5, but rather than using a double-stranded linker as shown for Example 5, a single-stranded linker which folds back upon itself can be used. In this example, the linker is: 5′-GGTCACACAGGAAACAGCTATGACAAACCCCGTCATAGCTGTTTCCTGTGTGA-3′ (SEQ ID NO:10).

In SEQ ID NO:10, the first 24 bases are the same as SEQ ID NO:7. The next 7 bases are a region which is capable of forming a loop. The final 22 bases are complementary to bases 24-3 of SEQ ID NO:7 (equivalent to SEQ ID NO:8) thereby allowing the molecule to fold back upon itself to form a double-stranded molecule with a loop, the GG at the 5′-end remaining single-stranded. This molecule can anneal to and be ligated with the Fau I digested microbial DNA. The loops can optionally be digested with DNAse specific for single-stranded DNA, e.g., mung bean nuclease (New England Biolabs, Inc., Beverly, Mass.). Amplification via PCR is performed using 5′-GTCATAGCTGTTTCC-3′ (SEQ ID NO:9) as the primer.

### EXAMPLE 7

Although the use of double-stranded linkers is preferable as stated in Example 2, use of a single-stranded oligomer which does not fold back upon itself can be used with Fau I in a manner similar to what was done in Example 1 in which BstF5 I was used. However, because the two enzymes leave different types of overhangs, the procedure will be slightly different.

As in Example 5 an unknown microorganism is cultured, nucleic acid is extracted and purified, and the nucleic acid is digested according to the supplier's recommended conditions by Fau I (New England Biolabs, Inc., Beverly, Mass.). This digest leaves 2-base 5′-overhangs. In this Example, a single-stranded oligomer is used. The single-stranded oligomer is 5′-GGTCACACAGGAAACAGCTATGAC-3′ (SEQ ID NO:7). This single-stranded oligomer will anneal to fragments which have CC dimer ends as 5′ overhangs. This oligomer is ligated to the digested fragments. Fragments with ligated oligomers are then amplified by PCR using a primer which is complementary to all or part of the single-stranded oligomer. In this Example, the primer is 5′ -GTCATAGCTGTTTCCTGTGTGA-3′ (SEQ ID NO:8). The amplified fragments are then analyzed as described in Example 5.

Those of skill in the art will recognize that other linkers with different ends can be used and various combinations of linkers and enzymes can be used, the invention not being limited to the linkers, primers and enzymes of the Examples. While the invention has been disclosed in this patent application by reference to the details of preferred embodiments of the invention, it is to be understood that the disclosure is intended in an illustrative rather than in a limiting sense, as it is contemplated that modifications will readily occur to those skilled in the art, within the spirit of the invention and the scope of the appended claims.

### LIST OF REFERENCES

Beyermann B, et al. (1992). Theor. Appl. Genet. 83:691-694.

Brenner S and Livak K J (1989). Proc. Natl. Acad. Sci. U.S.A. 86:8902-8906.

Caetano-Anolles G, et al. (1991). Bio/Technology 9:553-557.

Edwards A, et al. (1991). Am. J. Hum. Genet. 49:746-756.

Innis M A, et al. (1990). PCR Protocols: A Guide to Methods and Applications (Academic Press, San Diego).

Jeffreys A J, et al. (1985). Nature 314:67-73.

Jeffreys A J, et al. (1991). Nature 354:204-209.

McDowell D (1999). “PCR: Factors Affecting Reliability and Validity” in Analytical Molecular Biology Quality and Validation, eds. G C Saunders and H C Parkes (The Royal Society of Chemistry, Cambridge, UK), pp. 58-80.

Nakamura Y, et al. (1987). Science 235:1616-1622.

Ripabelli G, et al. (2000). System. Appl. Microbiol. 23:132-136.

Saunders G C and Hopkins D (1999). “PCR: Factors Affecting Reliability and Validity” in Analytical Molecular Biology Quality and Validation, eds. G C Saunders and H C Parkes (The Royal Society of Chemistry, Cambridge, UK), pp. 103-122.

Tautz D (1989). Nucleic Acids Res. 17:6463-6472.

Vos P, et al. (1995). Nucl. Acids Res. 23:4407-4414.

Welsh J and McClelland M (1990). Nucleic Acids Res. 18:7213-7218.

Welsh J and McClelland M (1991). Nucleic Acids Res. 19:861-866.

Willems A, et al. (2000). System. Appl. Microbiol. 23:137-147.

Williams J G K, et al. (1990). Nucleic Acids Res. 18:6531-6535.

Patents

EP 0535858

U.S. Pat. No. 5,508,169

U.S. Pat. No. 5,858,656

U.S. Pat. No. 5,874,215

What is claimed is:

1. A method of genotyping an organism, said method comprising the steps of:
(a) digesting genomic DNA of said organism with a restriction endonuclease, wherein said restriction endonuclease cleaves said genomic DNA at a site different from a recognition sequence site for said restriction endonuclease, wherein said restriction endonuclease cleaves said genomic DNA to produce DNA fragments having staggered ends;
(b) ligating said DNA fragments to linkers wherein said linkers have an end complementary to a staggered end of a portion of said DNA fragments to produce DNA fragments with linkers ligated at each end of a portion of said DNA fragments;
(c) amplifying said DNA fragments with linkers ligated at each end to produce amplified DNA fragments;
(d) separating the amplified DNA fragments by size;
(e) detecting each amplified DNA fragment wherein 5-100 fragments are detected; and
(f) determining a size for each detected DNA fragment.

2. The method of claim 1 further comprising:
(g) comparing the size of each DNA fragment against a database of restriction fragments of known organisms wherein a size match of each DNA fragment from said unknown organism with each size fragment of only one organism in said database identifies said unknown organism as said known organism.

3. The method of claim 1 wherein only one restriction endonuclease is used.

4. The method of claim 1 wherein only one linker is used.

5. The method of claim 4 wherein said linker is a single-stranded oligomer.

6. The method of claim 4 wherein said linker is partially double-stranded.

7. The method of claim 4 wherein said linker is comprised of a single DNA strand capable of internal basepairing to form a DNA structure that is partially double-stranded.

8. The method of claim 1 wherein only one primer is used.

9. The method of claim 1 wherein after performing said method in a first round said method is repeated for a second round with one or more variables changed as compared to the variables as used in the first round, wherein said variables are restriction endonucleases or linkers, further wherein data obtained from said first round and said second round are combined and analyzed.

10. The method of claim 1 wherein two linkers are used wherein a first linker has a staggered end different from a staggered end of a second linker.

11. The method of claim 10 wherein only one primer is used.

12. The method of claim 1 wherein said restriction endonuclease has a five base recognition sequence.

13. The method of claim 1 wherein said staggered ends consist of a dinucleotide extension.

14. The method of claim 1 wherein said restriction endonuclease is BstF5 I or Fau I.

15. The method of claim 1 wherein said amplified DNA fragments comprise a label.

16. The method of claim 15 wherein said label is a fluorescent dye.

17. The method of claim 1 wherein said organism is a microorganism.

18. The method of claim 17 wherein said microorganism is a bacterium, a mycobacterium, a yeast or a virus.

19. The method of claim 2 wherein said step of comparing is computerized.

20 The method of claim 1 wherein said separating is performed by gel electrophoresis.

21. A method of comparing the genomes of two microorganisms to determine whether the two microorganisms are the same or different, comprising:
a) digesting genomic DNA of each of said organisms with a restriction endonuclease, wherein said restriction endonuclease cleaves said genomic DNA at a site different from a recognition sequence site for said restriction endonuclease, wherein said restriction endonuclease cleaves said genomic DNA to produce DNA fragments having staggered ends;
b) ligating said DNA fragments to linkers wherein said linkers have an end complementary to a staggered end of a portion of said DNA fragments to produce DNA fragments with linkers ligated at each end of a portion of said DNA fragments;
c) amplifying said DNA fragments with linkers ligated at each end to produce amplified DNA fragments;
d) separating by size the amplified DNA fragments from each microorganism;
e) detecting the amplified DNA fragments which were separated by size; and
f) determining a size of each amplified DNA fragment;
wherein if the two microorganisms give different patterns of bands from each other the two microorganisms are a different species and wherein if the two microorganisms give an identical pattern of fragment sizes they are either the same species or two very closely related species.

22. The method of claim 21 wherein said separating is performed by gel electrophoresis.

23. A kit comprising (i) a restriction endonuclease wherein said restriction endonuclease is one which cleaves DNA at a site different from a recognition sequence site for said restriction endonuclease and wherein cleavage of genomic DNA by said restriction endonuclease produces DNA fragments having staggered ends and (ii) a linker wherein an end of said linker is complementary to a staggered end of a genomic DNA digested by said restriction endonuclease.

24. The kit of claim 23 further comprising (iii) a DNA ligase.

25. The kit of claim 23 further comprising (iii) a DNA polymerase.

26. The kit of claim 25 wherein said DNA polymerase is resistant to inactivation at a temperature below 70° C.

27. The kit of claim 24 further comprising (iv) a DNA polymerase.

28. The kit of claim 23 wherein said restriction endonuclease is selected from the group consisting of BstF5 I and Fau I.


SEQUENCE LISTING (10 sequences)

SEQ ID NO: 1 - DNA, 11 bp, Artificial Sequence
  Recognition site for Bgl I
gccnnnnnggc

SEQ ID NO: 2 - DNA, 11 bp, Artificial Sequence
  Recognition site for Fau I
nnnnnngcggg

SEQ ID NO: 3 - DNA, 24 bp, Artificial Sequence
  Synthetic oligomer
tcacacaggaaacagctatgacgg

SEQ ID NO: 4 - DNA, 22 bp, Artificial Sequence
  Synthetic oligomer
tcacacaggaaacagctatgac

SEQ ID NO: 5 - DNA, 22 bp, Artificial Sequence
  One strand of synthetic linker
gtcatagctgtttcctgtgtga

SEQ ID NO: 6 - DNA, 24 bp, Artificial Sequence
  Synthetic oligomer
tcacacaggaaacagctatgaccc

SEQ ID NO: 7 - DNA, 24 bp, Artificial Sequence
  One strand of synthetic linker
ggtcacacaggaaacagctatgac

SEQ ID NO: 8 - DNA, 22 bp, Artificial Sequence
  One strand of synthetic linker
gtcatagctgtttcctgtgtga

SEQ ID NO: 9 - DNA, 15 bp, Artificial Sequence
  Synthetic oligomeric primer
gtcatagctgtttcc

SEQ ID NO: 10 - DNA, 53 bp, Artificial Sequence
  Synthetic oligomeric linker
ggtcacacaggaaacagctatgacaaaccccgtcatagctgtttcctgtgtga